(19) United States
(12) Patent Application Publication
(10) Pub. No.: US 2016/0145552 A1
(43) Pub. Date: May 26, 2016

(54) FLOATING PHOTOBIOREACTOR SYSTEM COMPRISING A FLOATING PHOTOBIOREACTOR AND AN INTEGRATED PADDLE WHEEL AND AN AIRLIFT AND METHODS OF USE

(71) Applicants: George Philippidis, Tampa, FL (US); Andreas Michael Meise, Saarbrucken (DE); Lawrence Albert Walmsley, New York, NY (US); Michael Welch, Tampa, FL (US)

(72) Inventors: George Philippidis, Tampa, FL (US); Andreas Michael Meise, Saarbrucken (DE); Lawrence Albert Walmsley, New York, NY (US); Michael Welch, Tampa, FL (US)

(21) Appl. No.: 14/901,688

(22) PCT Filed: Jun. 24, 2014
(86) PCT No.: PCT/US2014/043765

§ 371 Date: Dec. 28, 2015

Related U.S. Application Data
(60) Provisional application No. 61/840,024, filed on Jun. 27, 2013.

(51) Int. Cl. C12M 1/00; C12M 1/09

(52) U.S. Cl. CPC C12M 21/02; C12M 23/26; C12M 23/56; C12M 29/08

(57) ABSTRACT

This invention relates to a floating photobioreactor system comprising a floating photobioreactor and an integrated paddle wheel and an integrated airlift. This invention further relates to methods of using the floating photobioreactor system.

### CROSS REFERENCE TO RELATED APPLICATION

This application claims the benefit U.S. Provisional Application No. 61/840,024, filed Jun. 27, 2013, which is incorporated by reference herein in its entirety.

### FIELD OF THE INVENTION

This invention relates to a floating photobioreactor system comprising a floating photobioreactor and an integrated paddle wheel and an airlift. This invention further relates to methods of using the floating photobioreactor system.

### BACKGROUND OF THE INVENTION

The majority of today's algae production is practiced in open systems (e.g., “open ponds”). These open systems are susceptible to contamination by foreign algae, parasites or other organisms. Thus, only algae with very specific growth characteristics can be cultivated in open systems. For example, the species Dunaliella grows in extremely salty water, in which hardly any other organisms can grow, and as such, it can be cultivated in an open system. In addition to being susceptible to contamination, open systems demonstrate low productivity. This results in a high production cost of algae. They also achieve usually only low biomass densities which results in high cost for pumping/treating the required water, harvesting and downstream processing. Depending on the area of application, the production costs may be too high and not economical. For example, the production costs of algae for the use in the energy sector are too high to be profitable.

As an alternative to open systems, a large number of closed systems (“photobioreactors”) have been developed. These include horizontal, flat photobioreactors, tubular photobioreactors and vertical, flat photobioreactors. Many of these photobioreactors are less susceptible to contamination and can reach higher productivities than open systems. However, photobioreactors have investment costs that are too high for many applications to achieve an economical production of algae biomass.

One example of a horizontal photobioreactor is a horizontal film reactor which can float on a pool of water. See, e.g., JP9001 182 and WO 2008/079724. In general, these horizontal film reactors have low investment cost, low susceptibility to contamination and good productivities. The low investment cost is due, in part, to the fact that these reactors can be manufactured using a low cost plastic film, e.g., polyethylene (“PE”). PE films can be processed easily by heat welding. See, e.g., WO 2008/079724. The resulting structure is flexible, which can facilitate the mixing of the system. See, e.g., JP9001 182.

While the flexibility of the horizontal reactor gives rise to a number of advantages, it also creates some challenges.

A major challenge is keeping a steady flow of water in the reactor (mixing). The constant movement of the algae solution is required to avoid settling of the algae, to avoid thermal stratification and to provide mixing for sufficient nutrient access by the algae.

In general, there are two major methods used for mixing: 1. Use of paddle wheels or any other impeller; and 2. Airlift pump or any other way to blow air or any gas mixture into the culture liquid to create a liquid movement.

Paddle wheels are used with a number of open pond designs. Paddle wheels are fixed at the ground and the media culture circulates in a non-flexible, non-floating, not-changing structure (the “pond”).

In addition to mixing, aeration could be another challenge in a flexible reactor. However, a good aeration, i.e. supply of CO₂ as a carbon source required for the growth of algae and potentially the removal of oxygen, is crucial to achieve high productivity. In general, three major means of aeration exist: 1) airlift pumps; 2) internal bubbling (bubbling of air into the reactor at one or several locations); and 3) semi-permeable diaphragm.

To keep the culture media moving and to aerate the media paddle wheels and airlift pumps have been suggested. Due to the technical challenges no paddle wheel has been previously suggested for a floating reactor system. In the same sense no integrated airlift has been previously proposed for an airlift reactor.

As described above, an external airlift for floating reactors has been suggested (U.S. Pat. No. 4,868,123). However, this represents an external airlift pump. The advantages and challenges to create an internal airlift pump for mixing and/or aeration (supply of CO₂ and removal of oxygen) into a floating photobioreacthor have been laid out above.

The second method for aeration is described by Meiser & Walmsley (Patent WO2010121136). They describe mixing and aeration through an inner aeration system by bubbling air through the floating reactor. This system has many advantages, overall low capital cost and achieving high productivities.

The third method for aeration is over a semi-permeable diaphragm. Such diaphragms would be too costly for using in a low-cost photobioreactor. Moreover, while semi-permeable diaphragms can be used to supply CO₂ to the algae suspension, it is unclear how they would remove oxygen from it.

In general, many pump system have been suggested for floating systems, however, these systems have a high energy consumption. Using a paddle wheel in a floating reactor system can decrease the energy consumptions.

In general, external airlift photobioreactors achieve a good mass transfer of CO₂ and oxygen between the gaseous and liquid phases. However, external aeration is not practical for mass production of algae. When used on a large scale with large volumes of algae suspension, the process becomes very energy consuming and costly. This is because large volumes of algae suspension must be continuously removed from the reactor and, following aeration, the suspension must be returned to the reactor. Additionally, external airlift pumps present a separate piece of equipment further increasing the costs. Furthermore, when an airlift pump is used in a tubular photobioreactor, the size of the photobioreactor is limited by the length of the tube for photosynthetic activity. Since the amount of oxygen increases over the length of the tube and the amount of CO₂ decreases, the maximum length of a tube in a tubular reactor is about 80 m. If the tubes are replaced by panels (horizontal, laminar reactors), it is possible to use a larger volume of algae suspension.

### SUMMARY OF THE INVENTION

The present invention provides a floating photobioreactor system that will be mixed by an integrated paddle wheel and aerated by an integrated airlift. In particular, the present invention provides a floating photobioreactor system comprising a floating photobioreactor and with a paddle wheel and an airlift.

The present invention also provides methods of using the floating photobioreactor system. The floating photobioreactor system may be used to grow photosynthetic or mixotrophic organisms. Examples for photosynthetic organisms could be microalgae, macroalgae, cyanobacteria, other photosynthetic active bacteria or even higher plants, such as duckweed. Alternatively, the floating photobioreactor system may be used to produce a biomass, a biofuel or a product selected from biochemicals, amino acids, fine chemicals, nutriceuticals, pharmaceuticals, energy products, protein, feed for cattle, fish and other species, protein source for human nutrition and mineral rich food for human consumption.

### BRIEF DESCRIPTION OF THE FIGURES

FIG. 1 is a three-dimensional view of a floating photobioreactor system with an integrated paddle wheel to provide mixing.

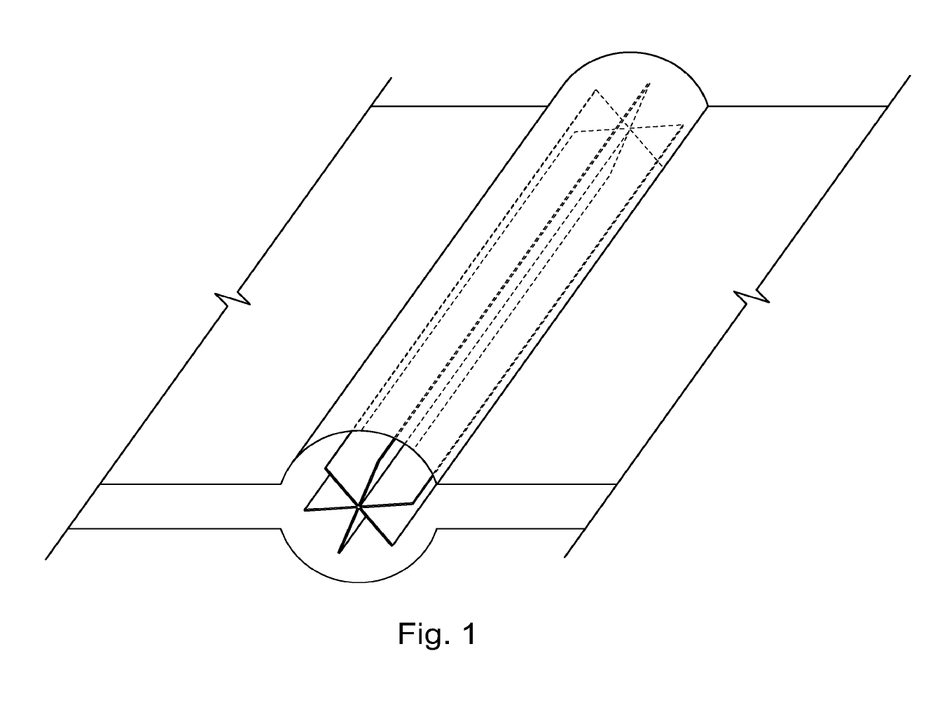

FIG. 2 is a three-dimensional view of a floating photobioreactor system with an integrated airlift to provide aeration.

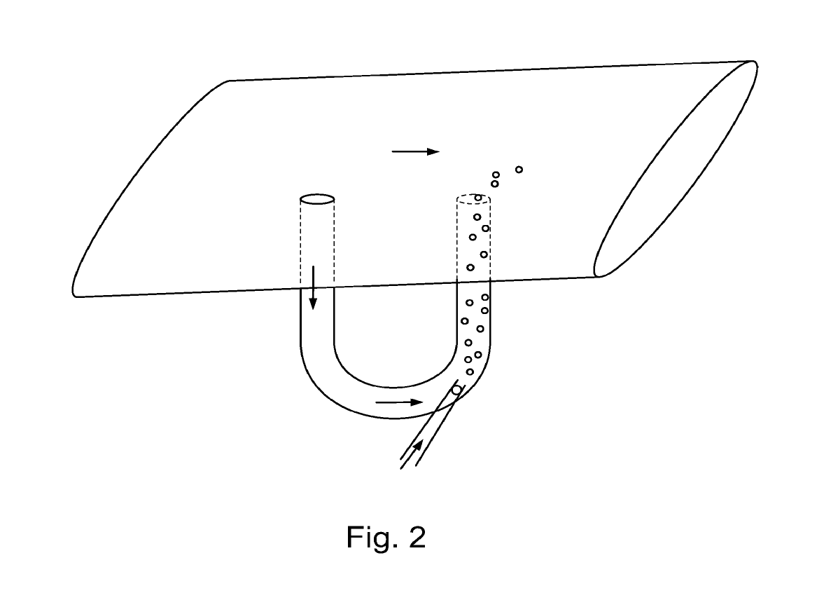

FIG. 3 is a cross section of a cutout of counter-flow aeration integrated in the photobioreactor system

FIG. 4 is a top view of the floating photobioreactor system which includes the paddlewheel and inlets and outlets

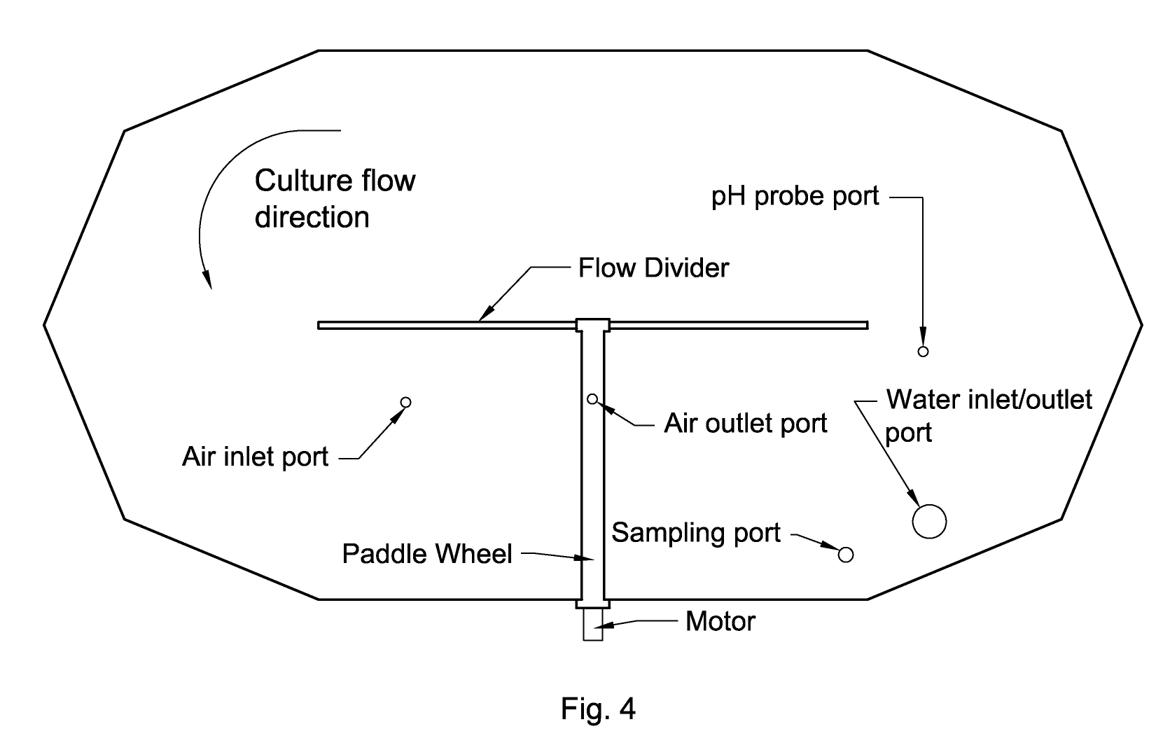

FIG. 5 is a side view of the floating photobioreactor system which includes the paddlewheel

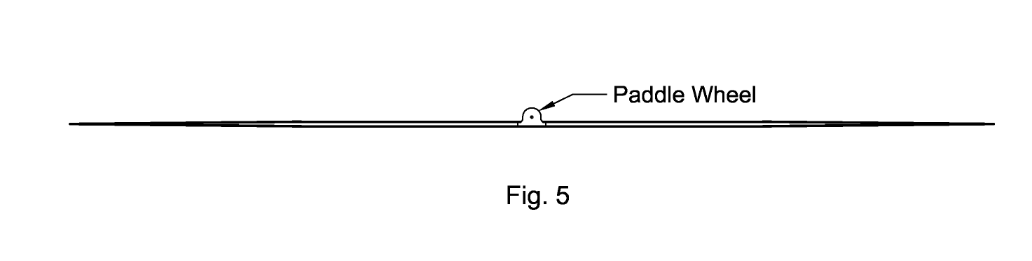

### DETAILED DESCRIPTION OF THE INVENTION

In order that the invention herein described may be fully understood, the following detailed description is set forth.

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

Throughout this specification, the word “comprise” or variations such as “comprises” or “comprising” will be understood to imply the inclusion of a stated integer or groups of integers but not the exclusion of any other integer or group of integers.

The term “a” or “an” may mean more than one of an item.

The terms “and” and “or” may refer to either the conjunctive or disjunctive and mean “and/or”.

The term “about” means within plus or minus 10% of a stated value. For example, “about 100” would refer to any number between 90 and 110.

The term “aeration” includes the supply of CO₂ and the removal of oxygen at the same time, even if not both functions are always mentioned explicitly.

The present invention provides a floating photobioreactor system with an integrated paddle wheel and an airlift.

The advantages of integrating a paddle wheel into a floating, flexible reactor are multiple:
A paddle wheel provides an energy-efficient means of mixing the media in the floating reactor
By integrating the paddle wheel into the reactor fewer parts will be required, thus lowering the capital and maintenance costs.
By integrating the paddle wheel into the reactor the algae suspension does not need to be transported out of and into the reactor, thus saving energy and cost.
It will be easier to achieve an even flow in the reactor and to avoid “dead zones”.

The advantages of integrating an airlift into a floating, flexible reactor are multiple:
The airlift provides an energy-efficient means of mixing the media in the floating low cost reactor.
The airlift will represent a means to achieve a good mass transfer between gas and liquid phase, especially for CO₂ and oxygen. One or more airlifts can be operated in a counter flow mode to further increase the mass transfer.
By integrating the airlift into the reactor fewer parts will be required, thus lowering the capital and maintenance costs.
By integrating the airlift into the reactor the algae suspension does not need to be transported out of and into the reactor, thus saving energy and cost.
It will be easier to achieve an even flow in the reactor and to avoid “dead zones”.
The airlift will be in the water, i.e. inner and outer water pressure will balance out and a thinner and cheaper material can be used to build the airlift system.
The airlift will be in the water and will thus increase the surface area that is available to cool the reactor.
The airlift built from a rigid material can serve as an anchor to fix the position of the floating flexible reactor.

The present invention provides a floating photobioreactor system where an integrated paddle wheel provides enhanced mixing, an integrated airlift supports enhanced mixing and provides enhanced mass transfer for CO₂ and supports the removal of oxygen from the photobioreactor system. Parts of the photobioreactor system can be flexible.

The paddle wheel will be positioned such that the liquid level in the paddle wheel (solution of the photosynthetic or mixotrophic organism) will be the same as in the floating part of the photobioreactor. This will allow the paddle wheel to be integrated into a flexible photobioreactor without forcing all water to the paddle wheel section or all water into the floating part of the photobioreactor. (The paddle wheel itself might be floating—however, the floating part of the photobioreactor refers here to the photobioreactor system without the paddle wheel).

The airlift pump will be positioned such that the liquid level in the airlift (solution of the photosynthetic or mixotrophic organism) will be the same as in the floating part of the photobioreactor. This will allow the airlift to be integrated into a flexible photobioreactor without forcing all water to the airlift or all water into the floating part of the photobioreactor. (The airlift itself might be floating—however, the floating part of the photobioreactor refers here to the photobioreactor system without the airlift).

The floating part of the photobioreactor might have various widths (usually between 40 cm and 50 meters). To allow the paddle wheel to be fully integrated, the connection between the floating part of the photobioreactor and the paddle wheel section might be similar at least in one dimension. In addition, the floating part of the photobioreactor might be directly connected to the paddle wheel section without any hose or tube in between.

The floating part of the photobioreactor might be directly connected to the airlift without any hose or tube in between. This is in strong contrast to many other designs of external airlift, where the airlift pump has a more or less cylindrical shape (see Acién Fernandez et al., “Airlift-driven external-loop tubular photobioreactors for outdoor production of microalgae: assessment of design and performance,” Chemical Engineering Science 56 (2001), U.S. Pat. No. 4,868,123. By having a similar shape with the floating part of the photobioreactor, the airlift can be fully integrated not requiring any additional hoses or tubes.

In one embodiment, the integrated paddle wheel is constructed in such a way that it is floating by itself. The construction allows the paddle wheel to have the exact right buoyancy to float in the required position. By design, it can be constructed in such a way that is keeps the right buoyancy when it is working (moving the paddle wheel generating a angular movement) and when it is switched off (no angular movement generated). In one embodiment this can be achieved by having compartments with a lower density than the surrounding water connected to the paddle wheel. They will prevent the paddle wheel from sinking. In addition, a heavy object could be attached to the lower part of the paddle wheel which will keep it in position in case it becomes too buoyant.

In one embodiment, the integrated airlift is constructed in such a way that it is floating by itself. The construction allows the airlift to have the exact right buoyancy to float in the required position. By design, it can be constructed in such a way that is keeps the right buoyancy when it is working (with aeration turned on—increasing its buoyancy) and when it is switched off (no aeration—buoyancy decreases). In one embodiment this can be achieved by having compartments with a lower density than the surrounding water at the upper part of the airlift. They will prevent the airlift from sinking even when no aeration is present. In addition, a heavy object could be attached to the lower part of the airlift which will keep it in position in case it becomes too buoyant.

The floating part of the photobioreactor and the paddle wheel section might have a similar shape at least in one dimension and might be made from different material with even very different characteristics, e.g. flexible material vs. rigid material. The connection between the flexible and rigid part might be created by gluing together the two parts or by using clamps or any other connection.

The floating part of the photobioreactor and the airlift might be made from different material with even very different characteristics, e.g. flexible material vs. rigid material. The connection between the flexible and rigid part might be created by gluing together the two parts or by using clamps or any other connection.

The floating part of the photobioreactor and the paddle wheel might have a different life-time. The connection between them might be constructed in such a way that the part with the shorter life-time, e.g., the flexible part, can be replaced easily.

The floating part of the photobioreactor and the airlift might have a different life-time. The connection between them might be constructed in such a way that the part with the shorter life-time, e.g., the flexible part, can be replaced easily.

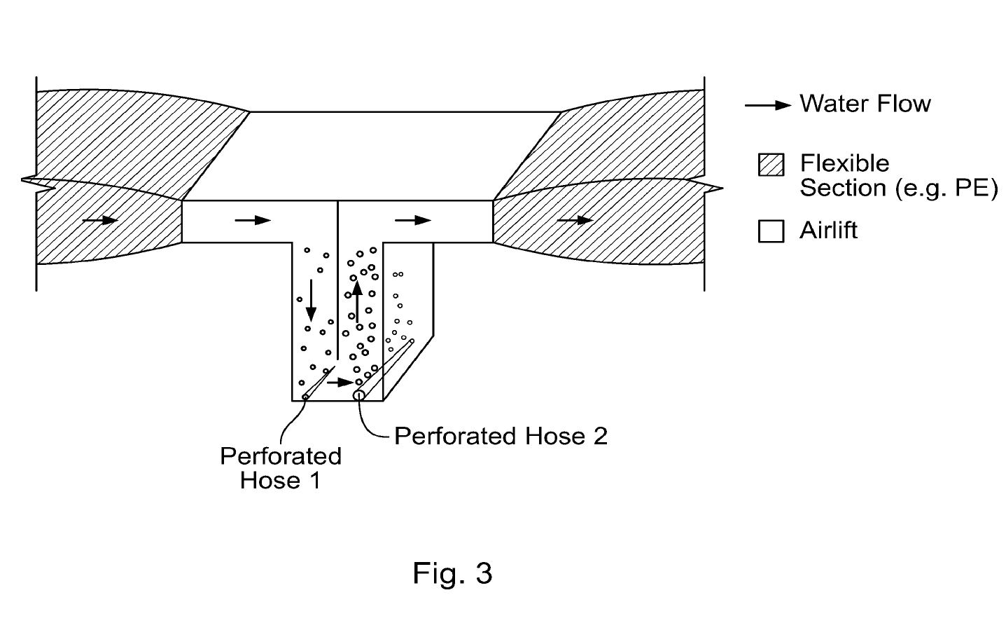

Airlift and a counter-flow aeration could also be combined. FIG. 3 shows one embodiment. In this embodiment the water would flow from left to right. At or near the bottom of the right section, bubbles of air, or any gas, potentially containing CO₂ will be blown into the liquid (coming out of Perforated hose 2). This will create a flow from left to right. At the left vertical chamber a CO₂ containing gas or even pure CO₂ is blown into the reactor by Perforated hose 1 to provide at least some of the CO₂ required for algae growth. The gas flow rate in the right vertical chamber (Perforated hose 2) might be higher to keep the water current flowing from left to right. There might be additional devices in the photobioreactor integrated to provide more mixing, e.g. a paddle wheel.

In one embodiment of the invention rigid structure will be introduced into the flexible part of the photobioreactor system. These rigid structures might change the shape of the flexible part, might prevent the flexible part or parts of it to move into any direction or might change the flow of the current or the gas streams. Examples might be a rigid frame which pushes the lower reactor sheet deeper into the surrounding water body, i.e. it increases the total height of the reactor system at one point or a certain area. Such an area which increased depth can be used to introduce CO₂—the additional depth improves the mass transfer of CO₂. Another example would be a structure that prevents that upper and lower sheet come too close and impact the current in a negative way.

The present invention provides methods of growing photosynthetic or mixotrophic organisms. According to the method, a suspension comprising the organism is introduced into one of the floating photobioreactor systems of the present invention. The photobioreactor is located in a surrounding water body. The suspension is exposed to light and brought into contact with a gas mixture comprising CO₂ and other nutrients.

The present invention also provides methods of producing biomass. According to this method, a suspension comprising the photosynthetic or mixotrophic organisms is introduced into one of the floating photobioreactor systems of the present invention. The photobioreactor is located in a surrounding water body. The organisms are grown in a suspension in the photobioreactor. The suspension is exposed to light and brought into contact with a gas mixture comprising CO₂ and other nutrients. The organisms produce a biomass, which is then harvested. The biomass may be harvested by methods known in the art.

The present invention also provides methods of producing a biofuel. According to this method, a suspension comprising the photosynthetic or mixotrophic organisms is introduced into one of the floating photobioreactor systems of the present invention. The photobioreactor is located in a surrounding water body. The organisms are grown in a suspension in the photobioreactor. The suspension is exposed to light and brought into contact with a gas mixture comprising CO₂ and other nutrients. The organisms produce a biomass, which is then harvested. Lipids, carbohydrates, proteins, vitamins, antioxidants, components from the photosynthetic or mixotrophic organism, and other components from the biomass are converted into biofuel. The conversion may be performed by methods known in the art.

The present invention also provides methods of producing a product selected from the group consisting of biochemicals, amino acids, fine chemicals, nutriceuticals, pharmaceuticals, energy products (ethanol, methane, hydrogen, fatty acids, fats and other lipids, highly energetic compound, propanol, butanol, gasoline-like fuel, diesel-like fuel, alkanes, alkenes, alcohols, organic acids, aromatic compounds), protein, feed for cattle or other species, fish feed, including feed for fish larvae and feed for other potential aquaculture uses, e.g., food for shrimps, crabs, oysters and their larvae, protein source for human nutrition and mineral rich food for human consumption. According to this method, a suspension comprising the photosynthetic or mixotrophic organisms is introduced into one of the floating photobioreactor systems of the present invention. The photobioreactor is located in a surrounding water body. The organisms are grown in a suspension in the photobioreactor. The suspension is exposed to light and brought into contact with a gas mixture comprising CO₂ and other nutrients. The organisms produce a biomass, which is then harvested. Lipids, carbohydrates, proteins, vitamins, antioxidants, components from the photosynthetic or mixotrophic organism, and other components from the biomass are converted into the desired product. The conversion may be performed by methods known in the art.

While particular materials, formulations, operational sequences, process parameters, and end products have been set forth to describe this invention, they are not intended to be limiting. Rather, it should be noted by those ordinarily skilled in the art that the written disclosures are exemplary only and that various other alternatives, adaptations, and modifications may be made within the scope of the present invention. Accordingly, the present invention is not limited to the specific embodiments illustrated herein.

What is claimed is:

1. A floating photobioreactor system to grow a photosynthetic or mixotrophic microorganism comprising:
a floating photobioreactor; and
an paddle wheel, wherein:
the paddle wheel is integrated into the floating photobioreactor.

2. The floating photobioreactor system according to claim 1, wherein the photobioreactor comprises a flexible material.

3. The floating photobioreactor system according to claim 1, wherein the paddle wheel comprises a rigid material.

4. The floating photobioreactor system according to any one of claims 1 to 3, wherein the photobioreactor is substantially horizontal.

5. The floating photobioreactor system according to any one of claims 1 to 4, further comprising a microorganism solution, which comprises a photosynthetic or mixotrophic microorganism and a growth medium.

6. The floating photobioreactor system according to claim 5, wherein the paddle wheel is capable of mixing the microorganism solution.

7. The floating photobioreactor system according to any one of claims 1 to 6, wherein the paddle wheel section has at least one dimension that is substantially similar in size to one dimension of the photobioreactor.

8. The floating photobioreactor system according to any of the claims 1 to 7, wherein the paddle wheel is directly connected to the photobioreactor without a hose or tube in between.

9. The floating photobioreactor system according to any of claims 6, 7 and 8, wherein the paddle wheel is floating.

10. The floating photobioreactor system according to any of claims 1 to 9, wherein the level of the microorganism solution inside the paddle wheel section is substantially the same as that inside the photobioreactor.

11. The floating photobioreactor system according to any of claims 1-10, further comprising a water body.

12. The floating photobioreactor system according to claim 11, wherein the photobioreactor system further comprises one or more additional compartments, each having a density different from the water body.

13. The floating photobioreactor system according to claim 12, wherein one additional compartment has a density higher than the water body and a second additional compartment has a density lower than the water body at the paddle wheel.

14. The floating photobioreactor system according to claim 12 or 13, further comprising a heavy object attached to the lower part of the paddle wheel.

15. The floating photobioreactor system according to any one of claims 1 to 8, wherein the paddle wheel is connected to a fixed object.

16. The floating photobioreactor system according to claim 15, wherein the fixed object is the ground of the water body or a wall.

17. The floating photobioreactor system according to any one of claims 1 to 16, wherein the paddle wheel is connected to the photobioreactor via glue, clamps or the like.

18. The floating photobioreactor system according to any one of claims 1 to 17, further comprising a counterflow aeration system.

19. The floating photobioreactor system according to any one of claims 1 to 18, further comprising a means for introducing gas into the flexible part of the photobioreactor.

20. The floating photobioreactor system according to claim 19, wherein the means for introducing gas is a perforated hose.

21. The floating photobioreactor system according to claim 20, wherein the gas comprises CO₂.

22. The floating photobioreactor system according to any one of claims 1 to 21, further comprising one or more additional paddle wheel.

23. A method of growing a photosynthetic or mixotrophic organism comprising:
(a) introducing a suspension comprising the photosynthetic or mixotrophic organism and growth medium into the floating photobioreactor system of any one of claims 1-22, wherein the photobioreactor is located in a surrounding water body;
(b) exposing the suspension to light; and
(c) contacting the suspension with a gas mixture comprising CO₂.

24. A method of producing a biomass comprising:
(a) growing a photosynthetic or mixotrophic organism in a growth medium in the floating photobioreactor system of any of claims 1-22, wherein the photobioreactor is surrounded by a water body;
(b) harvesting the biomass.

25. A method of producing a biofuel comprising:
(a) growing a photosynthetic or mixotrophic organism in a growth medium in the floating photobioreactor system of any one of claims 1-22, wherein the photobioreactor is surrounded by a water body;
(b) harvesting the organism; and
(c) converting one or more selected from the group consisting of lipids, carbohydrates, proteins, vitamins, or antioxidants from the organism, and components of the organism into the biofuel.

26. A method for producing a product comprising:
(a) growing a photosynthetic or mixotrophic organism in a growth medium in the floating photobioreactor system of any one of claims 1-22, wherein the photobioreactor is surrounded by a water body;
(b) harvesting the organism; and
(c) converting one or more selected from the group consisting of lipids, carbohydrates, proteins, vitamins, or antioxidants from the organism and components of the organism into the product,
wherein the product is selected from the group consisting of biochemicals, amino acids, fine chemicals, nutriceuticals, pharmaceuticals, energy products, protein, feed for cattle or other species, fish feed, protein source for human nutrition and mineral rich food for human consumption.

27. A floating photobioreactor system to grow a photosynthetic or mixotrophic microorganism comprising:
a floating photobioreactor; and
an airlift, wherein:
the airlift comprises an upper part and a lower part and
the airlift is integrated into the floating photobioreactor.

28. The floating photobioreactor system according to claim 27, wherein the photobioreactor comprises a flexible material.

29. The floating photobioreactor system according to claim 27, wherein the airlift comprises a rigid material.

30. The floating photobioreactor system according to any one of claims 27 to 29, wherein the photobioreactor is substantially horizontal.

31. The floating photobioreactor system according to any one of claims 27 to 30, further comprising a microorganism solution, which comprises a photosynthetic or mixotrophic microorganism and a growth medium.

32. The floating photobioreactor system according to claim 31, wherein the airlift is supporting the mixing and is capable of aerating and degassing the microorganism solution.

33. The floating photobioreactor system according to any one of claims 27 to 32, wherein the airlift has at least one dimension that is substantially similar in size to one dimension of the photobioreactor.

34. The floating photobioreactor system according to any of the claims 27 to 33, wherein the airlift is directly connected to the photobioreactor without a hose or tube in between.

35. The floating photobioreactor system according to any of claims 32, 33 and 34, wherein the airlift is floating.

36. The floating photobioreactor system according to any of claims 27 to 35, wherein the level of the microorganism solution inside the airlift is substantially the same as that inside the photobioreactor.

37. The floating photobioreactor system according to any of claims 27-36, further comprising a water body.

38. The floating photobioreactor system according to claim 37, wherein the photobioreactor system further comprises one or more additional compartments, each having a density different from the water body.

39. The floating photobioreactor system according to claim 38, wherein one additional compartment has a density higher than the water body and a second additional compartment has a density lower than the water body at the upper part of the airlift.

40. The floating photobioreactor system according to claim 38 or 39, further comprising a heavy object attached to the lower part of the airlift pump.

41. The floating photobioreactor system according to any one of claims 27 to 34, wherein the airlift pump is connected to a fixed object.

42. The floating photobioreactor system according to claim 41, wherein the fixed object is the ground of the water body or a wall.

43. The floating photobioreactor system according to any one of claims 27 to 42, wherein the airlift is connected to the photobioreactor via glue, clamps or the like.

44. The floating photobioreactor system according to any one of claims 27 to 43, further comprising a counterflow aeration system.

45. The floating photobioreactor system according to any one of claims 27 to 44, further comprising a means for introducing gas into the flexible part of the photobioreactor.

46. The floating photobioreactor system according to claim 45, wherein the means for introducing gas is a perforated hose.

47. The floating photobioreactor system according to claim 46, wherein the gas comprises CO₂.

48. The floating photobioreactor system according to any one of claims 27 to 47, further comprising one or more additional airlifts.

49. A method of growing a photosynthetic or mixotrophic organism comprising:
(a) introducing a suspension comprising the photosynthetic or mixotrophic organism and growth medium into the floating photobioreactor system of any one of claims 27-48, wherein the photobioreactor is located in a surrounding water body;
(b) exposing the suspension to light; and
(c) contacting the suspension with a gas mixture comprising CO₂.

50. A method of producing a biomass comprising:
(a) growing a photosynthetic or mixotrophic organism in a growth medium in the floating photobioreactor system of any of claims 27-48, wherein the photobioreactor is surrounded by a water body;
(b) harvesting the biomass.

51. A method of producing a biofuel comprising:
(a) growing a photosynthetic or mixotrophic organism in a growth medium in the floating photobioreactor system of any one of claims 27-48, wherein the photobioreactor is surrounded by a water body;
(b) harvesting the organism; and
(c) converting one or more selected from the group consisting of lipids, carbohydrates, proteins, vitamins, or antioxidants from the organism, and components of the organism into the biofuel.

52. A method for producing a product comprising:
(a) growing a photosynthetic or mixotrophic organism in a growth medium in the floating photobioreactor system of any one of claims 27-48, wherein the photobioreactor is surrounded by a water body;
(b) harvesting the organism; and
(c) converting one or more selected from the group consisting of lipids, carbohydrates, proteins, vitamins, or antioxidants from the organism and components of the organism into the product,
wherein the product is selected from the group consisting of biochemicals, amino acids, fine chemicals, nutriceuticals, pharmaceuticals, energy products, protein, feed for cattle or other species, fish feed, protein source for human nutrition and mineral rich food for human consumption.

53. The floating photobioreactor system according to claim 1, wherein a rigid structure is introduced in the flexible part. This rigid structure changes the shape of the flexible part, prevents the flexible part or parts of it to move into any direction or changes the flow of the current or the gas streams.

54. The floating photobioreactor system according to claim 27, wherein a rigid structure is introduced in the flexible part. This rigid structure changes the shape of the flexible part, prevents the flexible part or parts of it to move into any direction or changes the flow of the current or the gas streams.
